(19) United States
(12) Patent Application Publication
(10) Pub. No.: US 2016/0106315 A1
(43) Pub. Date: Apr. 21, 2016

(54) SYSTEM AND METHOD OF DIAGNOSIS USING GAZE AND EYE TRACKING

(71) Applicant: UMOOVE SERVICES LTD., Jerusalem (IL)

(72) Inventor: Yitzchak KEMPINSKI, Geva Binyamin (IL)

(21) Appl. No.: 14/723,590

(22) Filed: May 28, 2015

Related U.S. Application Data
(60) Provisional application No. 62/005,045, filed on May 30, 2014.

(51) Int. Cl. A61B 3/113; A61B 3/14; A61B 3/00; A61B 5/00; A61B 5/16

(52) U.S. Cl. CPC A61B 3/113; A61B 5/6898; A61B 5/168; A61B 5/4088; A61B 5/4082; A61B 3/0025; A61B 3/0041; A61B 5/4863; A61B 3/145

(57) ABSTRACT

A system and method for use of a mobile device to capture one or more images of one or more eyes of a user in a period before, during or after conducting one or more tests involving the eyes, vision or eye movements of the user. A position, motion, pattern or other characteristic of the position, motion or movement of one or more eyes may be evaluated in each of such images and comparisons of such positions, motions or movements in the various images may be made as part of a diagnostic effort for various medical conditions.

### CROSS-REFERENCE TO RELATED APPLICATIONS

This application claims priority of U.S. Provisional Patent Application No. 62/005,045, filed May 30, 2014, which is hereby incorporated by reference.

Eye tracking and gaze tracking are used in various modalities within the medical field. Listed below are certain patters of eye movements and some of the maladies associated with those eye movements. Eye movement tracking is useful in but not limited to the diagnosis, screening, management tracking, deterioration, detection characteristics associated therewith:
Saccadic Movements (ADHD, autisim)
anti-saccades, (Concussion)
self-paced saccades, (Concussion)
memory-guided sequences and smooth pursuit (Concussion)
Smooth pursuit
Preferential looking techniques (autism)
Spatial localization
Reciprocal social interactions (autism)
Microperimetry for oculomotor fixation
Saliency judgement (Itti, Koch, Nieber)
   Visual saliency models
   Contrast saliency models
View-invariant object category learning, recognition, and search
Order of fixation
Symmetrical movement and mode (mirror)
Optikinetic nystagmus (okin)
   Slow tracking and rapid recovery saccade
Predictive visual training (concussion)
Visual Paired Comparison (VPC) task for the detection of memory impairment associated with MCI (MCI->AD)
   saccade orientation, re-fixations and fixation duration
Visual Spatial (VSP) (Alzheimers)
   Early detection and longitudinal tracking
Convergence training (convergence insufficiency, ADHD)

Previously, eye or gaze tracking was performed by a qualified medical practitioner who may have watched the eyes of a patient. More recently, work stations or other fixed computer platforms have been used for tracking or monitoring the eyes or gaze of a patient in various circumstances.

### BRIEF DESCRIPTION OF THE DRAWINGS

FIG. 1 is a schematic drawing of a system in accordance with an embodiment of the invention.

### EMBODIMENTS OF THE INVENTION

Embodiments of the invention may enable eye and gaze tracking performed using a mobile electronic device to be used as a diagnostic indicator for one or more of the above and below listed conditions. In some embodiments, a method of gaze tracking may be as described in the document attached as Appendix A hereto entitled System and Method of Detecting a Gaze of a Viewer, in the document attached as Appendix B hereto entitled System and Method for Detecting Micro Eye Movements in Two Dimensional Image Captured with a Mobile Device or in the document attached as Appendix C hereto entitled Eye Tracking, and the documents attached hereto as Exhibit D.

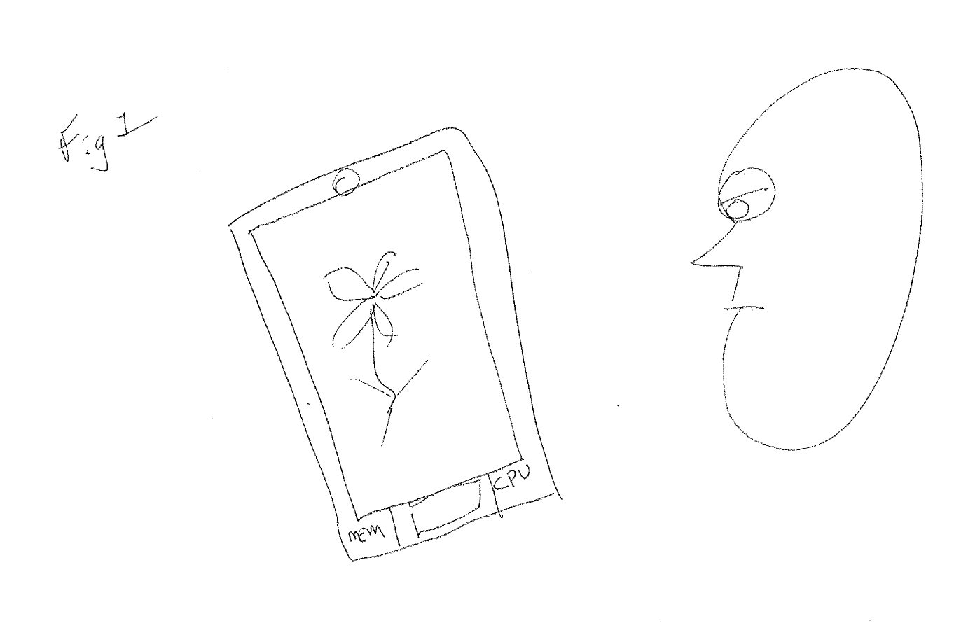

Embodiments of the invention may include a system for diagnosis a medical condition, where the system, a schematic illustration of which is shown no FIG. 1 includes an imager to capture several or a series of two dimensional images of at least one of an eye and head of a person using visible light. The system may include an electronic display at a known angle and position relative to the imager. The system may include a memory and a processor that is associated with the imager and the display. The imager, processor, memory and display may be included in a single mobile housing. The processor may collect data about at least one gaze point of a person appearing in the image as such person looks at the display in the series of image. The processor may compare the data about the person's gaze in such images with stored gaze data of persons with a condition selected from the group consisting of schizophrenia, attention deficit—hyperactive disorder, Amyotrophic lateral sclerosis, Concussion (mild Traumatic brain injury mTBI) (Post Concussive Syndrome PCS), autism, Optikinetic nystagmus and Alzheimer's Disease. Eye and gaze tracking may also be used in the treatment and management of diseases including vestibular disease and vertigo.

Alzheimer's Disease (AD)

Deficits in visuospatial cognition are pervasive in AD. Recent neurophysiological and imaging studies have revealed that changes in visuospatial perception (VSP) functions can be detected in the early stages of AD. Key advantages of utilizing VSP-related deficits in AD for early detection and longitudinal tracking of AD

We found that AD patients need significantly more time to initiate and execute goal-directed hand movements. AD patients are also unable to suppress reflexive eye and, to a lesser extent, hand movements. Furthermore, AD patients use a stepwise approach of eye and hand movements to touch a sequence of stimuli, whereas controls more often show an anticipatory approach. The impairments in reflex suppression of eye and hand movements, and changes in relative timing of eye-hand coordination, in AD patients support the notion that cortical networks involving the posterior parietal cortex are affected at an early disease-stage. It also suggests that the problems of AD patients to perform daily activities that require eye-hand coordination are not only caused by cognitive decline, but also by degeneration of neural networks involved in visuomotor coordination.

In summary, the results of this study indicate that machine learning methods can aid the automatic detection of cognitive impairment based on eye tracking data.

Autism

Location of gaze with high accuracy (precision <1 visual degree, sampling rate 50-300 Hz).

Looking, gazing, fixating, (stabilized gaze on fixed target) visual attention, eye movements, saccades, and smooth pursuit, Peripheral cues, saccadic latencies, oculomotor performance, visual orienting, action predicting and gaze following, predictive tracking.

Concussion (mild Traumatic brain injury mTBI) (Post Concussive Syndrome PCS)

Frequent lapses in attention are a characteristic symptom of TBI. Traditional measures that use discrete responses are unable to detect momentary lapses in attention.

Antisaccades tasks, a type of eye-movement paradigm sensitive to frontal lobe dysfunction, rely on discrete stimulus-response sets. Anti-saccades tasks may be useful once subjects have perceived PCS symptoms; however, this paradigm may not be sensitive to acute mTBI. Because attention varies over time, a relatively continuous measure of performance is needed to detect moment-to-moment fluctuations in attention within individuals. The examination of performance of visual tracking of a moving target may provide a supplement to conventional behavioral assessments of mTBI patients. Using video-oculography, eye movement can be monitored easily, precisely, and continuously. In contrast to the anti-saccade paradigm, visual tracking does not rely on discrete stimulus response sets during the maintenance phase. Visual tracking of a moving target requires the integration of multiple sensory inputs and one's own motor efforts. Visual tracking also requires cognitive processes including target selection, sustenance of attention, spatiotemporal memory, and expectation. Quantification of visual tracking performance using a circular target trajectory, provides a continuous behavioral assessment metric. The motion of a target traveling at a constant velocity with a fixed radius from the center is highly predictable. This movement can continue indefinitely within the orbital range of the eye, which makes the stimulus particularly suitable for studying the processes required to maintain predictive visual tracking. Predictive visual tracking requires both attention and working memory, processes for which the PFC is considered to be an important substrate. These cognitive functions are often compromised in mTBI patients. Visual tracking performance can be objectively quantified using parameters, such as smooth pursuit velocity gain, phase error, and root-mean square error. Because TBI is known to increase intra-individual performance variability on visuomotor tasks, we measured the variability of visual tracking performance in terms of gaze positional error relative to the target to grade the level of performance. Good visual tracking was characterized by overall tight clustering of the gaze positions around the target. In contrast, poor visual tracking in mTBI subjects was generally characterized by a wide distribution of the gaze along the circular path, which indicates spatio-temporal dyssynchrony with the stimulus. The spread of visual-tracking gaze errors (variability) can be used as an attention metric and can be correlated with an individual's degree of white matter integrity.

The Post Concussion Syndrome group performed worse on anti-saccades, self-paced saccades, memory-guided sequences and smooth pursuit, suggesting problems in response inhibition, short-term spatial memory, motor-sequence programming, visuospatial processing and visual attention.

Eye movement function in PCS does not follow the normal recovery path of eye movements after mCHI, marking ongoing cerebral impairment independently of patient self-report and neuropsychological assessment. Importantly, poorer oculomotor function was unrelated to depression or estimated IQ. Whilst oculomotor and neuropsychological tests partially overlapped in identifying suboptimal brain function, eye movements provided additional evidence of dysfunction in areas such as decision making under time pressure, response inhibition, short-term spatial memory, motor-sequence programming and execution, visuospatial processing and integration, visual attention and subcortical brain function. Indications of poorer subcortical/subconscious oculomotor function in the PCS group support the notion that PCS is not merely a psychological entity but also has a biological substrate. Eye movements might be of particular interest in PCS cases with high symptom load and poor ability to cope with activities of daily living but whose clinical test profile is otherwise unremarkable with regard to neuropsychological testing or other assessments. Eye movement testing, and evidence of suboptimal subcortical functioning in particular, may help demonstrate incomplete recovery of brain function in such cases. Despite the costintensive nature of eye movement assessment in terms of required equipment, eye movement testing should be feasible in centres, which have easy access to eye tracking technology.

ALS

“The study of oculomotor dysfunction holds significant promise as an additional source of much needed prognostic, monitoring, and mechanistic biomarkers for ALS.”

ADHD

Convergance Insufficiency

Convergence insufficiency (CI) is a common and distinct binocular vision disorder that affects approximately 4% of school age children and adults in the United States. Convergence insufficiency is often associated with symptoms such as frequent loss of place while reading, loss of concentration, having to re-read, reading slowly, poor comprehension, sleepiness, blurred vision, diplopia, headaches, and/or eyestrain. A recently completed randomized clinical trial, the Convergence Insufficiency Treatment Trial (CITT), demonstrated that a 12-week program of office-based vergence/accommodative therapy with home reinforcement was more effective than home-based near target pencil push-ups, home-based computer accommodative therapy plus pencil push-ups, or office-based placebo therapy in treating the symptoms and signs associated with symptomatic CI in children 9 to 17 years of age.

While the home-based therapies in the CITT were not as effective as office-based vergence/accommodative therapy there was some improvement noted. Currently, many eye care professionals only offer home-based therapy, while others suggest passive treatment with base-in prism. At a Pediatric Eye Disease Investigator Group (PEDIG) meeting (Tampa, January 2009), the results of a poll of attendees indicated that a large majority of pediatric ophthalmologists continue to recommend home-based near target push-ups as the initial treatment approach for children with symptomatic CI in spite of the CITT results.

Schizophrenia

Evidence for the specificity of SPEM dysfunction to diagnosed schizophrenia, as well as to healthy individuals with a genetic vulnerability to schizophrenia, suggests that the SPEM task has efficacy as a test of gene carrier status in schizophrenia, and therefore as a trait marker of risk for schizophrenia.

Pyschiatric Disease

A few studies addressed impaired smooth pursuit eye movements (eye tracking dysfunction) in unaffected relatives of psychiatric patients, and were important in excluding non-specific effects (e.g. medication) and isolating genetic predisposition to the disease. This predisposition could be demonstrated in families of schizophrenic patients irrespective of whether the index case was sporadic or familial. One large study demonstrated pathological distributions of various parameters of smooth pursuit eye movement performance in groups of schizophrenic patients and their relatives. However, another study challenged the specificity of eye tracking dysfunction as a trait marker for schizophrenia by showing that its prevalence was identical among relatives of patients with affective disorder and schizophrenia. Eye tracking dysfunction was associated with two gene polymorphisms that interfere with dopamine metabolism and are thus reasonable candidate genes for the predisposition to schizophrenia. The influence of nicotine and neuroleptic drugs on eye movement performance was studied in schizophrenic patients. Nicotine improved smooth pursuit performance in three studies, one of which attributed this finding to enhanced attention.

Eye movements provide an important tool to measure pharmacological effects in patients and unravel genetic traits in psychiatric disease.

Neurodegenerative Disease

Eye movement recordings in the laboratory are generally not necessary for diagnostic purposes, but can be a useful addition to the clinical examination. Laboratory recordings of eye movements can provide valuable information about disease severity, progression or regression in neurodegenerative disease, and hold particular promise for objective evaluation of the efficacy of putative neuroprotective and neurorestorative therapies.

Other ailments that may present or be detected by tracking of eye movements include Parkinson's Disease, Glaucoma, Fragile X Syndrome, Progressive Supranuclear Palsy, Attention, and Affective Disorder. Use of eye tracking and gaze tracking as a diagnostic or therapeutic method in some or all of such disorders is described on one or more of the papers attached hereto as Exhibit C.

Instructions for embodiments of the invention may be, for example, loaded by one or more processors and be executed by one or more processors from instructions stored in one or more computer readable mediums. For example, the computer readable medium may be a non-transitory computer readable storage medium. A non-transitory computer readable storage medium may be, for example, an electronic, optical, magnetic, electromagnetic, infrared, or semiconductor system, apparatus, or device, or any combination thereof. Computer program code may be written in any suitable programming language. The program code may execute on a single computer system, or on a plurality of computer systems.

1. A method of diagnosis of a medical condition, comprising:
tracking at least one of a movement of an eye of a patient and a movement of a gaze of said patient with an imager of a mobile electronic device using visible light; and
diagnosing from at least one of said movement of said eye of said patient and said gaze of said patient, a condition of said patient, said condition selected form the group consisting of schizophrenia, attention deficit—hyperactive disorder, amyotrophic lateral sclerosis, concussion (mild traumatic brain injury mTBI) (Post Concussive Syndrome PCS), autism, optikinetic nystagmus and Alzheimer's Disease, Parkinson's Disease, Glaucoma, Fragile X Syndrome.

2. A system for diagnosis a medical condition, said system comprising:
an imager, said imager to capture a plurality of two dimensional images of at least one of an eye and head of a person using visible light;
an electronic display, said display at a known angle and position relative to said imager;
a processor associated with said imager and said display
wherein said processor is to:
collect data about at least one gaze point of said user at said display in said plurality of images, and
compare said data with stored gaze data of persons with a condition selected from the group consisting of schizophrenia, attention deficit—hyperactive disorder, Amyotrophic lateral sclerosis, Concussion (mild Traumatic brain injury mTBI) (Post Concussive Syndrome PCS), autism, Optikinetic nystagmus and Alzheimer's Disease Parkinson's Disease, Glaucoma, and Fragile X Syndrome.
